(19) United States
(12) Patent Application Publication
(10) Pub. No.: US 2007/0107540 A1
(43) Pub. Date: May 17, 2007

(54) Method and apparatus for assessing or characterizing properties of powdered or particulate materials

(76) Inventor: Clive Eric Davies, Waikanae (NZ)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/499,565

(22) PCT Filed: Dec. 20, 2002
(86) PCT No.: PCT/NZ02/00289

§ 371 Date: Feb. 22, 2005

(30) Foreign Application Priority Data
Dec. 21, 2001 (NZ) 516328

(51) Int. Cl. G01N 33/00

(52) U.S. Cl. 73/866

(57) ABSTRACT

A method for assessing a property or properties of a powdered or particulate material, which includes: containing the material within, or causing the material to flow through, a rotating vessel or chamber supported via a sensor indicative of force or weight, monitoring an output signal from the sensor as the container or vessel rotates and the material within the container or vessel moves, and assessing the material property by reference to the output signal of the sensor. Apparatus for assessing material properties is also disclosed.

### FIELD OF INVENTION

The invention comprises a method and apparatus for assessing or characterizing properties of powdered or particulate materials.

### BACKGROUND

When a particulate solid or the like such as an assembly of particles or grains is constrained or contained in equipment that rotates, the motion or movement of the particles is affected by several factors. These include the geometry and configuration of the containing equipment, the speed of rotation, the properties and characteristics of the particles, the properties and characteristics of the assembly of particles, the characteristics and friction characteristics of the walls of the equipment, and whether the particulate solid or assembly of particles or grains are free flowing particles or grains or have some cohesiveness because of the nature or size of the particles or grains or because of the nature or action of fluid in contact with the particles or grains or the surface of the particles or grains.

In some equipment, in a circular drum or disc for example, the particulate solid or assembly of particles or grains may be moved in the direction of rotation and may slide or form sliding beds which travel from the wall of the rotating drum and fall along the sloping free surface of the powder bed contained or constrained in the rotating equipment, or may move in a dispersed state in the equipment. At least seven different modes or regimes of motion of the material in a rotating drum in the plane of a cross-section through the drum have been identified. J. Mellmann in a paper in Powder Technology, Volume 118, page 251-270, 2001, has termed these: sliding, surging, slumping, rolling, cascading, cataracting, and centrifuging. There can also be movement of material along an axis of the equipment.

A system for characterizing powder avalanching in a rotating drum is disclosed by B H Kaye in Powder Bulk Engineering, February 1996, which uses a light beam directed through a transparent rotating drum containing a powder sample and a photocell array positioned on the opposite side of the drum which is blocked to a greater or lesser degree as the powder avalanches within the drum. The output of the photocell array represents powder avalanching within the drum. A powder flowability analyzer marketed under the trade mark AERO-FLOW is commercially available from TSI Inc (www.tsi.com).

U.S. Pat. No. 5,847,294 discloses apparatus for determining flowability of powder by sensing powder avalanching within a rotating sample drum, which senses avalanching via a torque loading sensor arranged to sense variations in torque loading and drive motor or coupling mechanism.

### SUMMARY OF INVENTION

In broad terms the present invention in one aspect comprises a method for assessing a property or properties of a powdered or particulate material, which includes:
containing the material within, or causing the material to flow through, a rotating vessel or chamber supported via a sensor indicative of force or weight,
monitoring an output signal from the sensor as the container or vessel rotates and the material within the container or vessel moves, and
assessing the material property by reference to the output signal of the sensor.

The vessel or chamber may be supported by a structure which is pivotally mounted relative to a base on one side of the vessel or chamber and is supported via said sensor between the structure and the base on another side of the vessel. The vessel or chamber may be supported by a structure which is pivotally mounted on one side along an axis which is substantially parallel to a longitudinal axis of rotation of the vessel or chamber and which is substantially horizontal, and via a sensor between the structure and the base on another side of the longitudinal axis of the vessel.

Preferably the method includes assessing a property or properties of the material by reference to the variance in impulses or fluctuations in the output signal of the sensor.

Preferably the method includes assessing a property or properties of the material by obtaining from the output signal of the sensor information on movement of the material within the vessel or chamber about a reference point.

The property of the material may be the flowability of the material. Another property of the material may be a transition of the material from a surging behaviour to a slumping behaviour or vice versa. Another property of the material is a transition of the material from a slumping behaviour to a rolling behaviour or vice versa. Another property of the material is the relative particle size of the material.

In broad terms in another aspect the invention comprises apparatus for assessing a property or properties or a powdered or particulate material, which includes a vessel or chamber mounted for rotation and supported via a sensor indicative of force or weight and which provides output signal as the container or vessel rotates and the material within the container or vessel moves, and means for assessing the material property by reference to the output signal of the sensor.

Preferably the apparatus includes a computer processor arranged to assess a property or properties of the material by reference to the variance in impulses or fluctuations in the output signal of the sensor.

A computer processor may also be arranged to assess a property or properties of the material by extracting from the output signal of the sensor information on movement of the material within the vessel or chamber about a reference point.

The apparatus may be arranged to assess a property or properties of the material on-line as it flows continuously through the rotating vessel or chamber.

The apparatus may include a computer processor arranged to assess flowability of the material.

The apparatus may include a computer processor arranged to assess a transition of the material from a surging behaviour to a slumping behaviour or vice versa, of the material.

The apparatus may include a computer processor arranged to assess a transition of the material from a slumping behaviour to a rolling behaviour or vice versa of the material.

The apparatus may include a computer processor arranged to assess a relative particle size of the material.

The apparatus may be configured so that a specified amount of material is placed in the rotating vessel or chamber and remains in the vessel or chamber as it rotates. Alternatively the apparatus may be arranged so that there is a flow of material into the chamber or vessel and out of the vessel or chamber as it rotates. When conditions are steady, the flow into the equipment is the same as the flow out of the equipment, and the amount of material that is held up in the equipment will depend on a variety of factors including the size, geometry, inclination, speed of rotation, the properties and characteristics of the particles, the properties and characteristics of the assembly of particles, and the characteristics and friction characteristics of the particles and walls of the equipment, and the rate of flow into the equipment.

### BRIEF DESCRIPTION OF THE DRAWINGS

The method and apparatus of the invention are further described with reference to the accompanying drawings by way of example and without intending to be limiting, wherein:

FIG. 1 is a side view of one embodiment of apparatus of the invention,

FIG. 2 is a front view of the apparatus of FIG. 1,

FIG. 3 is a plan view of the apparatus of FIGS. 1 and 2,

FIG. 4 is a side view of another embodiment of an apparatus of the invention,

FIG. 5 is a front view of the apparatus of FIG. 4,

FIG. 6 is a plan view of the apparatus of FIGS. 4 and 5,

FIGS. 7 and 8 are schematic diagrams which illustrate the effect of movement of the material in the drum on the position of the center of gravity of the material in the drum,

FIGS. 9, 10 and 11 are plots of filtered weight variance versus rotation rate which show the effect of rotation rate on the output signal from a load cell, and are referred to further in the subsequent description of experimental work.

### DETAILED DESCRIPTION OF PREFERRED FORMS

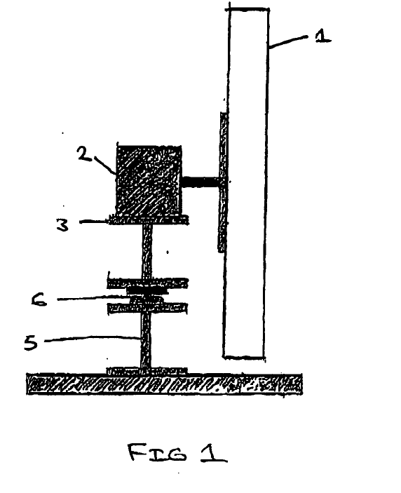

In its most general form and with reference to FIG. 1, the invention consists of a cylindrical vessel 1 which is caused to rotate by motive drive means which may consist of an electric motor 2 or the like, and if required a gearing system 2a and drive wheels or the like and other systems to ensure that the desired mode of rotation is achieved. The speed of rotation can be controlled.

The drum 1 and motive drive means 2 are mounted on a platform structure 3 which is pivoted about an axis along one side at bearings 4 or similar. Preferably the assembly is in turn carried on a base 5. A load cell 6 or often force or weight sensor 6 is located on the base 5 and is positioned in a way that the force arising from the moment due to the mass of the platform 3, the mass of the vessel 1 and the mass of the contents of the vessel can be measured. The position of the load cell or force measuring means can be varied.

The platform structure is preferably fitted with a counterweight 7 for counterbalancing the weight of the platform 3 and the vessel 1 and the motor 2 and drive means.

It will be appreciated that the size of the force or weight that is sensed by the sensor 6 will depend on the relative location of the load cell or force measuring means with respect to the pivot 4, as well as the weight of the platform 3 and items carried by it, and the distribution of the weight of the platform, that is to say the weight of the platform and the position of its centre of gravity, and the weight of any item on the platform and the position of the centre of gravity of material placed on the platform or in the cylindrical vessel. The size of the force that is measured will be affected by changes in the position of the centre of gravity of the material in the drum arising from the motion of the material, as the drum rotates.

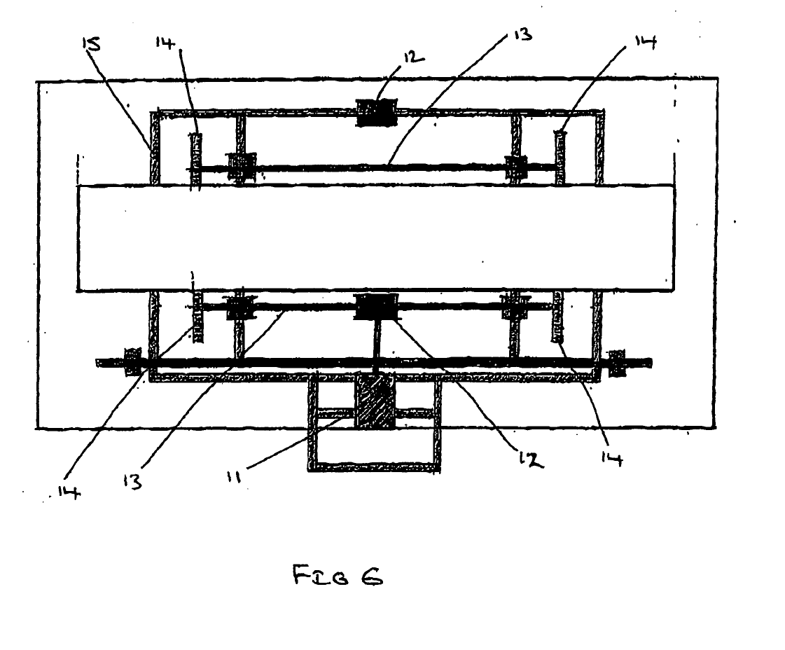

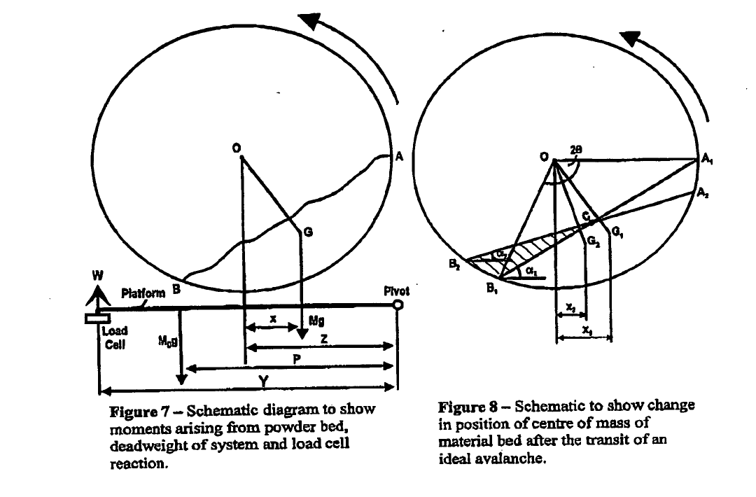

This can be better understood by considering FIGS. 7 and 8, where FIG. 7 is a schematic diagram which shows moments arising from powder bed, deadweight of system and load cell reaction, and FIG. 8 is a schematic diagram which shows the change in position of the centre of mass of the material bed after the transit of an ideal avalanche.

Considering the identities in FIG. 7, and taking moments about the pivot,
WY=Mg(Z−x)+M_{D}gP (1)
where W is the force sensed by the load cell; Y is the distance between the pivot and the load cell; M is the mass of powder in the cylinder; g is acceleration due to gravity; Z is the distance from the pivot to the centre line through the vertical axis of the drum; x is the horizontal distance from the centre line to the centre of mass, G; and M_{D} is the deadweight of the assembly acting through a point a horizontal distance P from the pivot. Note that if we rewrite Equation 1 with x≡( x̅+x′) where x̅ is the time averaged mean of x, and x′ is a transient which may take positive or negative values, then it follows that the variance of W is directly proportional to Σx′².

FIG. 8 shows how the passage or an ideal avalanche, shown as the cross-hatched wedge of material in FIG. 8, across the material bed changes the position of the centre of mass of the material in the bed. The positions of the centre of gravity of the material in the drum for the ideal avalanche before and after it moves across the cross section of the drum, are shown in FIG. 8 as x₁ and x₂ and likewise the angles of the free surface are given as α₁ and α₂.

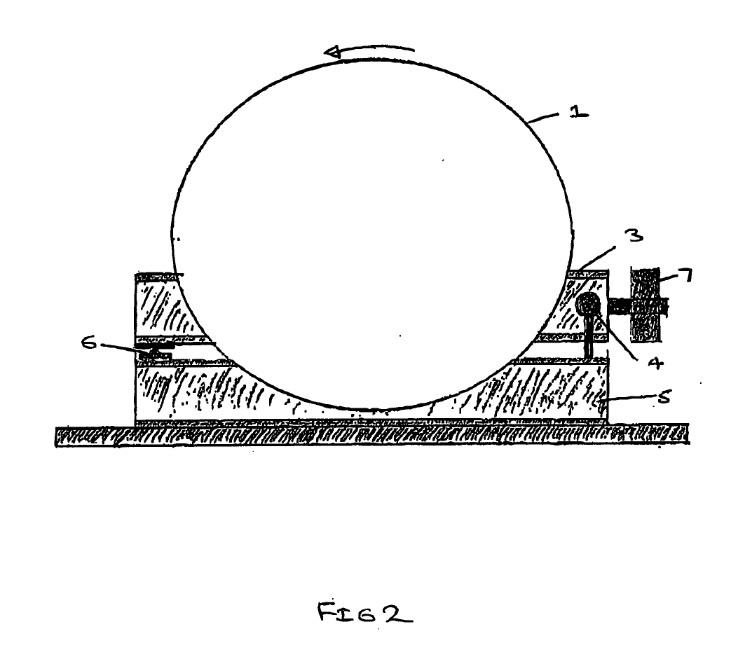

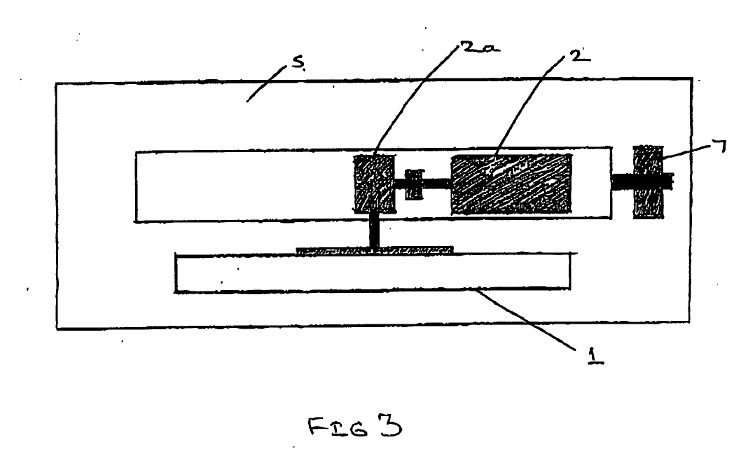

Prior to carrying out a test with a sample of material in the batch equipment depicted in FIGS. 1 to 3, the material which may be a known weight or volume of material, is placed in the vessel 1. The vessel equipment is caused to rotate, and the load cell output is monitored and recorded, over a period of time which is chosen to be long enough to enable sufficient data for characterization of the powder to be recorded. For example, for a speed of rotation of 15 revolutions per minute, the data could be recorded over a period of 20 minutes, or even over a period of one hour but in many circumstances, significantly shorter periods are sufficient.

The form of the apparatus shown in FIGS. 1 to 3 is well suited to characterization measurements in which the material under test is contained in a circular container for which the width or depth of the container is significantly smaller than the diameter.

For equipment that is operated continuously, the material is caused to flow through the equipment and measures are taken to ensure that the flow is steady.

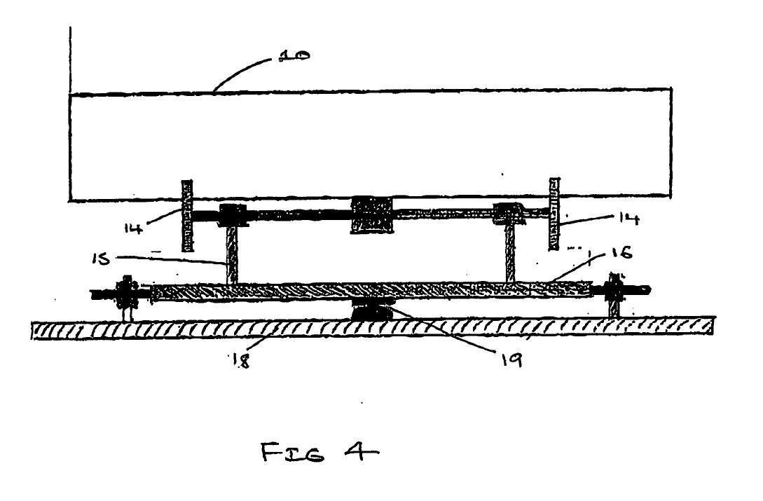

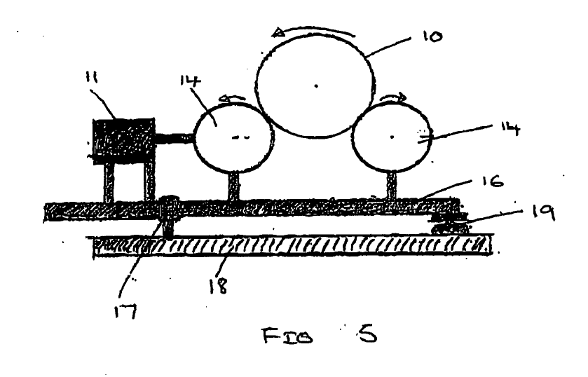

The apparatus of FIGS. 4-6 is similar to that of FIGS. 1 to 3 in principle except that the vessel into which the material is placed or through which material may continuously flow is of an elongated cylindrical shape as shown. The vessel 10 is supported by drive rollers 14 which are carried on the ends of shafts 13 mounted for rotation in a subframe 15. The subframe 15 also carries electric motor 11 which is connected to the drive shafts 13 carrying drive rollers 14 through gear boxes 12. The subframe 15 carrying the cylinder 10 and drive arrangement is in turn pivotally mounted on base 18 via pivot 17 and load cell 19. A counterweight forming a counterbalance (not shown) may be provided. It will be appreciated that there are alternative ways of constructing and assembling the apparatus and means for driving it.

As the cylinder 10 rotates, powdered or particulate material contained within the cylinder or flowing through the cylinder from one end to the other, in a production stream for example, will move as a result of the rotational motion of the drum in a way that causes fluctuations in the output signal of the load cell 19.

For all forms of the apparatus, information on the movement and motion and characterization of the material in the rotating equipment of the invention can be obtained in a number of ways, and some examples are given without intending to be limiting. In one approach the output signal of the load cell is recorded preferably in digital form, and impulses or fluctuations in the output signal, due to movements of the material under test as the container or vessel rotates, are analysed. The signal may be processed to give the statistical parameter, variance, or the related statistical parameter, standard deviation. These statistical parameters can be compared with the values obtained for reference materials and used to make a judgment on the characteristics and properties and motion and mode of motion, on the basis of experience with the reference materials, or can be used with reference to a correlation of material characteristics and properties with variance or standard deviation.

In another approach, a quantity or batch of material is placed in the rotating equipment of the invention, and general equations which can be derived by considering the shape of the powder bed, and taking moments about a reference point, the position of the centre of gravity of the mass of powder can be determined at any instant from the output of the load cell. By way of example only, an idealised shape for the powder bed when the rotating equipment is a circular drum or a disc shaped container that rotates slowly, is the shape of a segment of a circle. Changes in the position of the centre of gravity of this idealised shape at successive intervals of time, can be interpreted as being the result of movement of material from one part of the ideal segment shape to another part of the segment shape as the equipment rotates; the amount of material that moves can be calculated using the measurements of the ideal shape of the powder bed, and the position of the centre of gravity that is found from the load cell output. When the rotating equipment of the invention is operated for a period of time, information on the movement of material can be calculated and recorded, and can be used to characterize the material by comparison with the behavior of a reference material. This comparison can be carried out using statistical techniques, or by using statistical parameters such as variance or standard deviation. Alternatively, different materials can be compared directly by comparing the characteristic information for each material.

### EXPERIMENTAL

The invention is further illustrated by the following description of test results, which is given by way of example.

Apparatus was constructed in accordance with FIGS. 4 to 6, having a cylinder made of Perspex, at length L 300 mm, and diameter D was 150 mm. The cylinder was rotated by the action of rollers on a shaft driven by an electric motor with a power rating of 55 Watts and an integral gear box, with an output speed of approximately 25 rpm. Rotation rate was varied by controlling the motor speed with a X704 approximately 25 rpm controller by PDL Electronics, Napier, New Zealand, and could be varied over the range approximately 0.75 to approximately 25 rpm.

Apparatus was also constructed in accordance with FIGS. 1 to 3, having a cylinder comprising a narrow disc 130 mm in diameter and 25.4 mm deep, directly driven by a shaft threaded to the centre of an aluminum hub attached to the rear face of the disc. The front plate was transparent, to permit video imaging of the solids. The drive train consisted of a 24 volt DC motor with a maximum speed of 230 rpm and a 10:1 reducing right angle drive controller was used to change the motor speed, permitting operation in the range 0.6 to approximately 25 rpm.

In both systems, the deadweight of the drive assembly and framework could be reduced to any required level by an adjustable counterweight. This allows in principle the absolute range of the load cell used to be significantly lowered, so increasing the insensitivity of the instruments to small transients. The load cell used was a subminiature compression load cell, type 13/2443-06 by Sensotec, USA.

Results are presented for sago and for lactose. The sago had a weight mean diameter of 2.4 mm, and 94% by weight was in the size range 2-2.8 mm. The loose poured bulk density was 719 kg m⁻³. Three samples of lactose having mean diameters and densities as shown in Table 1, were prepared by sieving a bulk sample; the bulk sample had a mean diameter of 206 μm and a loose poured bulk density of 800 kg m⁻³.

TABLE 1
Bulk densities of different lactose materials.

| | Bulk Density |
|---|---|
| Material | (kgm⁻³) |
| 75-106 μm | 657.5 |
| 150-212 μm | 730.5 |
| 300-425 μm | 752.4 |

In the experiments with sago, the apparatus was charged to the required fill level, and rotated at the selected rate for approximately 240 seconds. The load cell signal was logged at 200 Hz to a PC. This signal was filtered using a Bartlett convolution filter, bandwidth=1 Hz, to remove all frequencies greater than 10 Hz, and then the variance calculated.

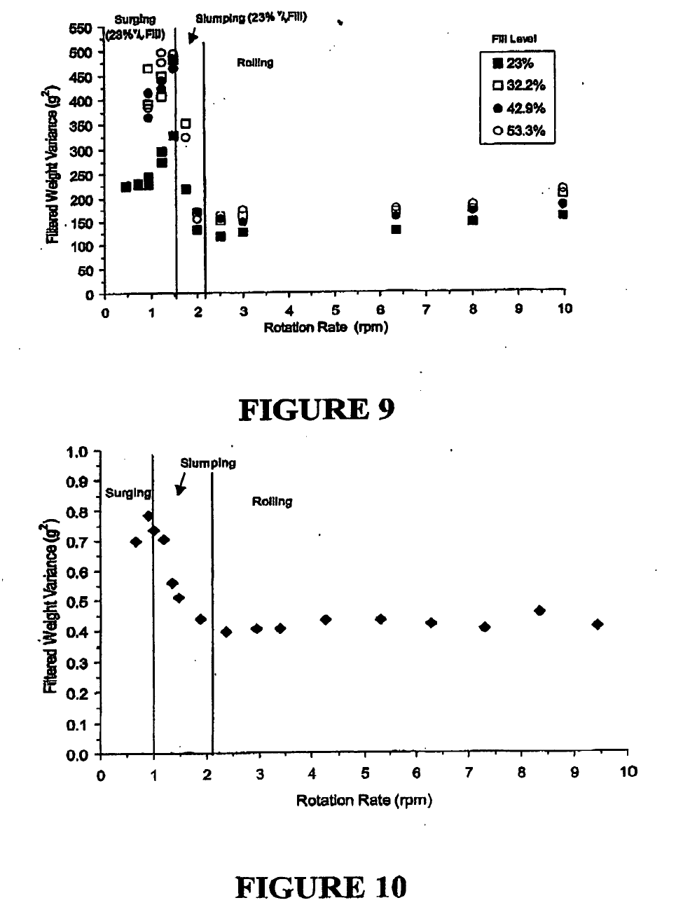

FIG. 9 shows the variance of the load cell signal, expressed in mass units, for the roller driven 300 mm cylinder, as a function of rotation rate in revolutions per minute, rpm, and fill level. The variance increases steeply as rotation rate increases, peaking around 1.1 to 1.5 rpm. As rotation rate is increased per, the variance rapidly falls, before levelling out to a value which is relatively constant for rotation rates of approximately 2 rpm and greater. Visual inspection, both by direct observation of the apparatus, and inspection of video footage of the particle motion at the end plate, indicated that the onset of the slumping regime occurs as the variance peaks; likewise, the transition to rolling takes place as the variance levels off.

FIG. 10 shows results obtained in the 130 mm diameter disc for a nominal fill of 20% v/v; again, the variance is expressed in mass units, and the rotation rate is rpm. The trend is the same as for the 300 mm cylinder, with the variance rising to a maximum at about 1 rpm, and then rapidly falling away to a steady value at approximately 2 rpm. Direct visual observation and video footage again confined the correlation of the surging/slumping and slumping/rolling transitions with the peak value of variance and the fall to a steady value.

The results in FIGS. 9 and 10 show the same trends, and demonstrate that the apparatus can be used to identify the transition through the surging mode to the slumping mode and then to the rolling mode, where these flow modes are the flow modes identified by J. Mellmann, in a paper in Powder Technology, Volume 118, page 251-270, 2001 The observed gross changes in the angle of the surface, α, in the slumping (avalanching) regime are manifested in larger values of the load cell variance than when α remains relatively constant as seen in the rolling regime.

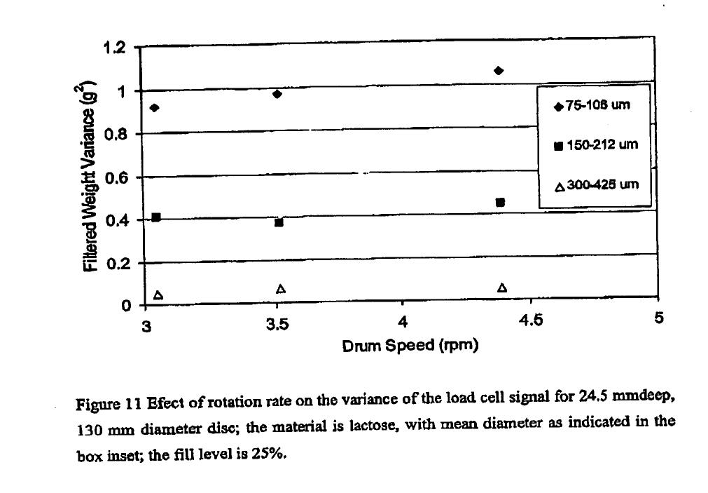

In experiments with lactose, the apparatus was charged to a fill level of 25% and data recorded over a 240 second period; frequencies greater than 3 Hz were filtered out. FIG. 11 shows values of variance plotted againse rotation rate for the different size fractions. It is apparent that the measured variance is higher for the material with the smaller mean particle size; at a given rotation rate, the measured variance decreases as the mean particle size of the material being tested increases.

The foregoing describes the invention including preferred thereof. Alterations and modifications as will be obvious to those skilled in the art are intended to be incorporated in the scope thereof as defined in the accompanying claims.

1. A method for assessing a property or properties of a powdered or particulate material, which includes:
containing the material within, or causing the material to flow through, a rotating vessel or chamber supported via a sensor indicative of force or weight,
monitoring an output signal from the sensor as the container or vessel rotates and the material within the container or vessel moves, and
assessing the material property by reference to the output signal of the sensor.

2. A method according to claim 1 wherein the vessel or chamber is supported by a structure which is pivotally mounted relative to a base on one side of the vessel or chamber and is supported via said sensor between the structure and the base on another side of the vessel.

3. A method according to claim 1 wherein the vessel or chamber is supported by a structure which is pivotally mounted on one side along an axis which is substantially parallel to a longitudinal axis of rotation of the vessel or chamber and which is substantially horizontal, and via a sensor between the structure and the base on another side of the longitudinal axis of the vessel.

4. A method according to any one of claims 1 to 3 including a counter-balancing at least in part the weight of the vessel and structure, on the other side of said pivot axis from the sensor.

5. A method according to any one of claims 1 to 4 including assessing a property or properties of the material by reference to the variance in impulses or fluctuations in the output signal of the sensor.

6. A method according to any one of claims 1 to 4 including assessing a property or properties of the material by obtaining from the output signal of the sensor information on movement of the material within the vessel or chamber about a reference point.

7. A method according to any one of claims 1 to 6 including assessing a property or properties of the material on-line as it flows continuously through the rotating vessel or chamber.

8. A method according to any one of claims 1 to 7 wherein the sensor indicative of force or weight is a load cell.

9. A method according to any one of claims 1 to 8 wherein the property of the material is the flowability of the material.

10. A method according to any one of claims 1 to 8 wherein the property of the material is a transition of the material from a surging behaviour to a slumping behaviour or vice versa.

11. A method according to any one of claims 1 to 8 wherein the property of the material is a transition of the material from a slumping behaviour to a rolling behaviour or vice versa.

12. A method according to any one of claims 1 to 8 wherein the property of the material is the relative particle size of the material.

13. Apparatus for assessing a property or properties of a powdered or particulate material, which includes a vessel or chamber mounted for rotation and supported via a sensor indicative of force or weight and which provides output signal as the container or vessel rotates and the material within the container or vessel moves, and means for assessing the material property by reference to the output signal of the sensor.

14. Apparatus according to claim 13 wherein the vessel or chamber is supported by a structure which is pivotally mounted relative to a base on one side of the vessel or chamber and is supported via said sensor between the structure and the base on another side of the vessel.

15. Apparatus according to claim 14 wherein the vessel or chamber is supported by a structure which is pivotally mounted on one side along an axis which is substantially parallel to a longitudinal axis of rotation of the vessel or chamber and which is substantially horizontal, and via a sensor between the structure and the base on another side of the longitudinal axis of the vessel.

16. A method according to any one of claims 13 to 15 including a counter-balance on the other side of said pivot axis from the sensor to counterbalance at least in part the weight of the vessel and structure.

17. Apparatus according to any one of claims 13 to 16 including a computer processor arranged to assess a property or properties of the material by reference to the variance in impulses or fluctuations in the output signal of the sensor.

18. Apparatus according to any one of claims 13 to 16 including a computer processor arranged to assess a property or properties of the material by extracting from the output signal of the sensor information on movement of the material within the vessel or chamber about a reference point.

19. Apparatus according to any one of claims 13 to 18 which is arranged to assess a property or properties of the material on-line as it flows continuously through the rotating vessel or chamber.

20. Apparatus according to any one of claims 13 to 19 wherein the sensor indicative of force or weight is a load cell.

21. Apparatus according to any one of claims 13 to 20 including an electric motor for driving rotation of the vessel or chamber.

22. Apparatus according to any one of claims 13 to 21 including a computer processor arranged to assess flowability of the material.

23. Apparatus according to any one of claims 13 to 21 including a computer processor arranged to assess a transition of the material from a surging behaviour to a slumping behaviour or vice versa.

24. Apparatus according to any one of claims 13 to 21 including a computer processor arranged to assess a transition of the material from a slumping behaviour to a rolling behaviour or vice versa.

25. Apparatus according to any one of claims 13 to 21 including a computer processor arranged to assess a relative particle size of the material.
